(19) United States
(12) Patent Application Publication
(10) Pub. No.: US 2022/0378620 A1
(43) Pub. Date: Dec. 1, 2022

(54) CONVENIENT SPORTS TAPE

(71) Applicant: Ashley Lee Kuo, Taipei City (TW)

(72) Inventor: Ashley Lee Kuo, Taipei City (TW)

(21) Appl. No.: 17/330,445

(22) Filed: May 26, 2021

(51) Int. Cl. A61F 13/00; A61F 13/02; C09J 7/25; C09J 7/24

(52) U.S. Cl. CPC A61F 13/00; A61F 13/0246; A61F 13/0259; A61F 13/00059; A61F 13/00076; C09J 7/255; C09J 7/245; C09J 7/243; A61F 2013/00578; A61F 2013/00604; C09J 2475/006; C09J 2467/006; C09J 2477/006; C09J 2429/006; C09J 2431/006; C09J 2427/006; C09J 2423/106; C09J 2423/046

(57) ABSTRACT

A convenient sports tape, comprising a package body and a tape body; the package body has at least one transparent top sheet, a bottom sheet matching the top sheet with its edge connected to that of the top sheet, and a housing space formed between the top sheet and the bottom sheet, the housing space can store and clamp the tape body; the tape body is provided with at least one adhesion section, and a positioning section configured on one end of the adhesion section; the tape body has three layers in total, made up of a tape layer, an adhesive layer, and a releasing layer of the same shape; the tape layer can be configured according to the marbling of lean flesh.

### BACKGROUND OF INVENTION

### 1. Field of the Invention

The present invention relates generally to a Kinesio tape, and more particularly to a convenient sports tape.

### 2. Description of Related Art

Sports tapes, also called Kinesio tapes, are a kind of elastic tape with no content of medicine. It is flexible and breathable. There is no need to remove the sports tapes after use. They can be adhered to the surface of the body and can be used in daily life. They are a protective tool with therapeutic and protective functions, used as a medical aid.

Based on the viscosity and elasticity of the sports tapes, as well as the principle of kinesiology and biomechanics, they can be used to strengthen or relax specific lean flesh. As they provide good function, they are widely used in recent years to prevent or cure sport hurt.

However, the conventional sports tapes are single-sheet rolls like film rolls. In usage and implementation, the user needs assistance from a third person or a medical professional to adhere them on the marbling of lean flesh at appropriate positions. It is quite difficult to use by one person only. Therefore, it is inconvenient to use.

In view of the above problem, the present invention aims to provide a convenient sports tape that can solve the above problem.

### SUMMARY OF THE INVENTION

The purpose of the present invention is to provide a convenient sport tape which is simpler and more convenient in application.

In order to solve the above problems and achieve the purpose of the present invention, the present invention is implemented by the following technical means:

A convenient sports tape, comprising a package body and a tape body; wherein the package body has at least one transparent top sheet, a bottom sheet matching the top sheet, with its edge connected to that of the top sheet, and a housing space formed between the transparent top sheet and the bottom sheet, the housing space can store and clamp the tape body; the tape body is provided with at least one adhesion section, and a positioning section configured on one end of the adhesion section; the tape body has three layers, made up of a tape layer, an adhesive layer, and a releasing layer of the same shape; the tape layer can be configured according to the marbling of lean flesh; the adhesive layer is configured on the bottom of the tape layer, and can be adhered to and positioned on the corresponding marbling of lean flesh of the user; the releasing layer is configured on the bottom of the adhesive layer in a detachable form.

More particularly, wherein the surface of the adhesion section and positioning section of the tape layer is configured with indicating or guiding labels.

More particularly, wherein the terminal of the free end of the adhesion section is formed with two or more branching sections.

More particularly, wherein the side of the adhesion section is formed with at least one extended section.

More particularly, the tape layer is made of a synthetic polymer, which can be any of the following materials: polyethylene (PE), polypropylene (PP), polyvinyl chloride (PVC), ethylene vinyl acetate copolymer (EVA), polyvinyl alcohol (PVA), polyamide (Nylon), thermoplastic polyurethane (TPU), polyethylene terephthalate (PET), or any combination of two or more than two of the above materials.

Comparing to the prior art, the present invention has the following effects and benefits:

1: In the present invention, as the convenient sports tape is made up of a package body and tape body, it is safer in terms of hygiene, and it allows quick and easy application by consumers, free of the difficulty to adhere a traditional single-roll sports tape to an appropriate position on the body.

Thus, the invention will make it easy for manufacturers to market their products.

2: In the present invention, as the surfaces of the adhesion section and positioning section are provided with indicative or guiding labels, consumers can apply the product according to the indication or guidance on the label. It makes easy for consumers and can avoid misplacement. Even without assistance of professionals, consumers can use the product by themselves with no confusion.

### BRIEF DESCRIPTION OF THE DRAWINGS

FIG. 1 is a perspective view of the sports tape according to first embodiment of the invention.

FIG. 2 is an exploded view of the sports tape according to first embodiment of the invention.

FIG. 3 is a usage view of the tape body according to first embodiment of the invention.

FIG. 4 is a usage view of the tape body according to second embodiment of the invention.

FIG. 5 is a usage view of the tape body according to third embodiment of the invention.

FIG. 6 is a perspective view of the tape body according to fourth embodiment of the invention.

FIG. 7 is a perspective view of the tape body according to fifth embodiment of the invention.

### DETAILED DESCRIPTION OF THE INVENTION

Below are detailed descriptions of the invention based on the embodiment depicted in the figures.

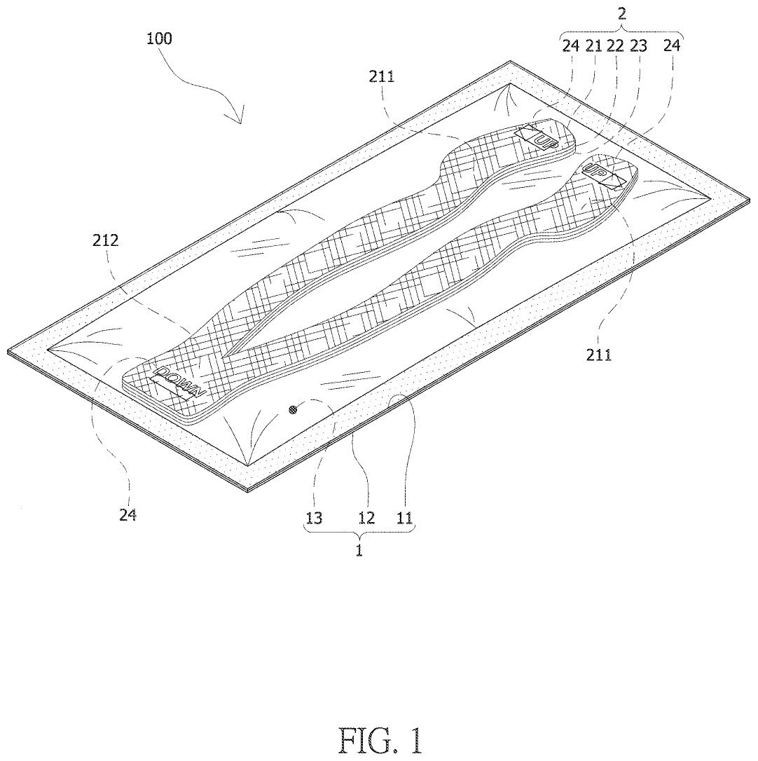

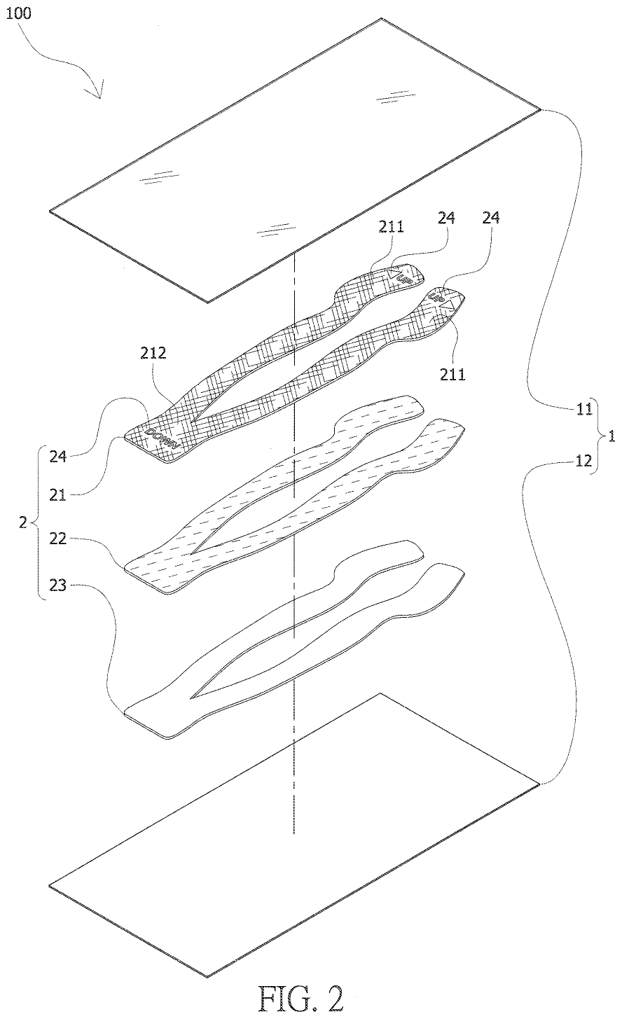

FIGS. 1 to 2 disclose a convenient sports tape 100, comprising a package body 1 and a tape body 2; the package body 1 has at least one transparent top sheet 11, a bottom sheet 12 matching the top sheet 11, with its edge connected to that of the top sheet 11, and a housing space 13 formed between the top sheet 11 and the bottom sheet 12, the housing space 13 can store and clamp the tape body 2; the tape body 2 is configured with at least one adhesion section 211, and a positioning section 212 formed on one end of the adhesion section 211; the tape body 2 has three layers in total, including a tape layer 21, an adhesive layer 22, and a releasing layer 23, all being of the same shape; the tape layer 21 can be configured according to the marbling of lean flesh; the adhesive layer 22 is configured on the bottom of the tape layer 21, and can be adhered to and positioned on the corresponding marbling of lean flesh of the user; the releasing layer 23 is configured on the bottom of the adhesive layer 22 in a releasable form.

The above-mentioned package body 1 can hold and protect the tape body 2 entirely, free of such problems as fall-off of the releasing layer 23 or contamination of the tape layer 21 before usage, and can therefore meet the requirements for hygiene and safety; as the top sheet 11 of the package body 1 is transparent, the tape body 2 clamped in the housing space 13 can be easily seen by naked eyes, and therefore consumers can freely select the type of tape body 2 they need. The adhesion section 211 and positioning section 212 of the tape body 2 are designed in different shapes to meet different sport needs by manufacturers. Thus, consumers can select different styles of convenient sports tapes 100 according to their personal needs and use them instantly, free of questions on how to operate or apply the product. Moreover, the adhesion section 211 is configured according to the marbling of lean flesh, and the positioning section 212 can provide basic positioning function.

To use the product, tear open the package body 1, take out the tape body 2, and then remove the releasing layer 23. Firstly, adhere the positioning section 212 to the appropriate body part of the consumer, then, extend the adhesion section 211 and stick it on the corresponding marbling of lean flesh. As the bottom side of the tape layer 21 has an adhesive layer 22, the product can be stably adhered to and positioned on the body part.

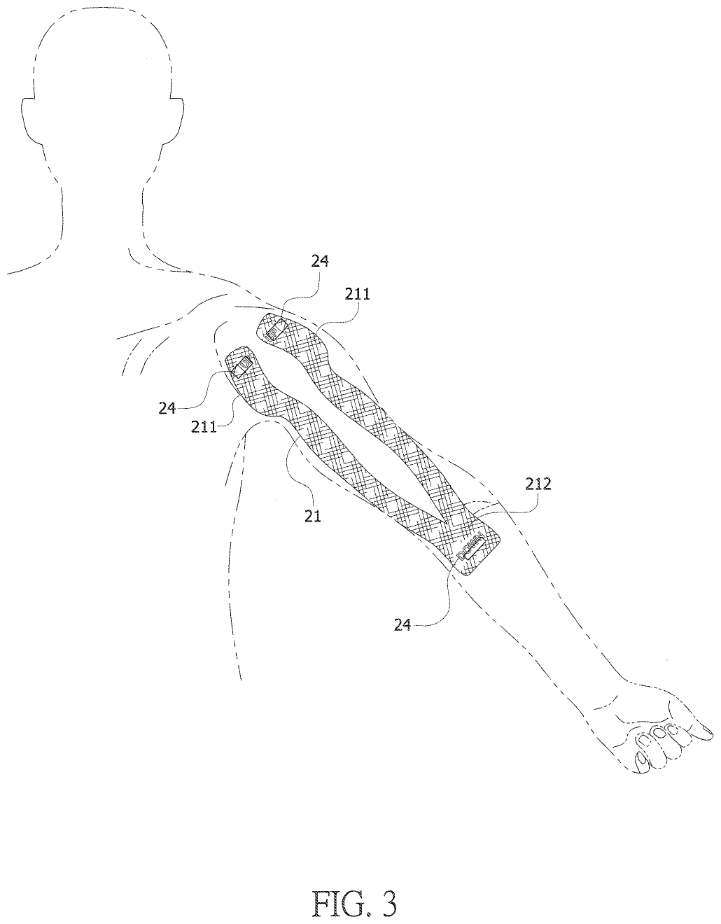

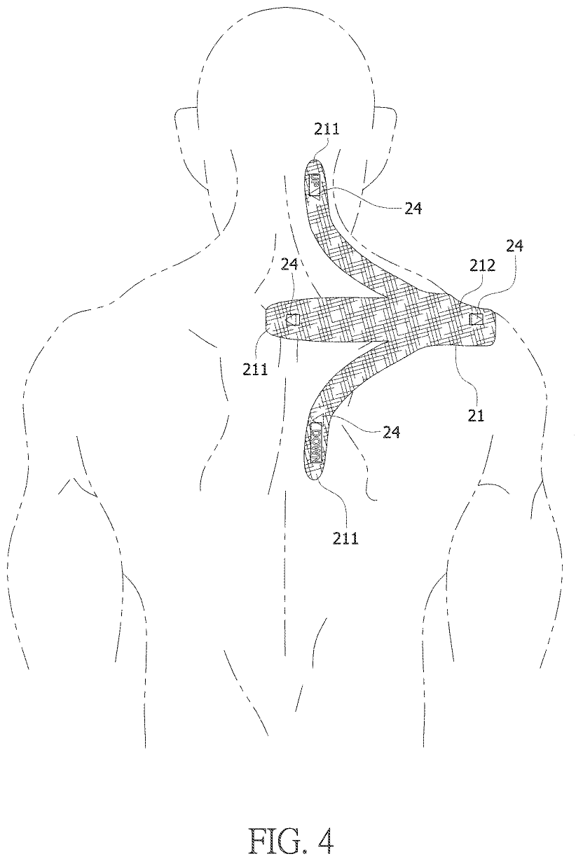

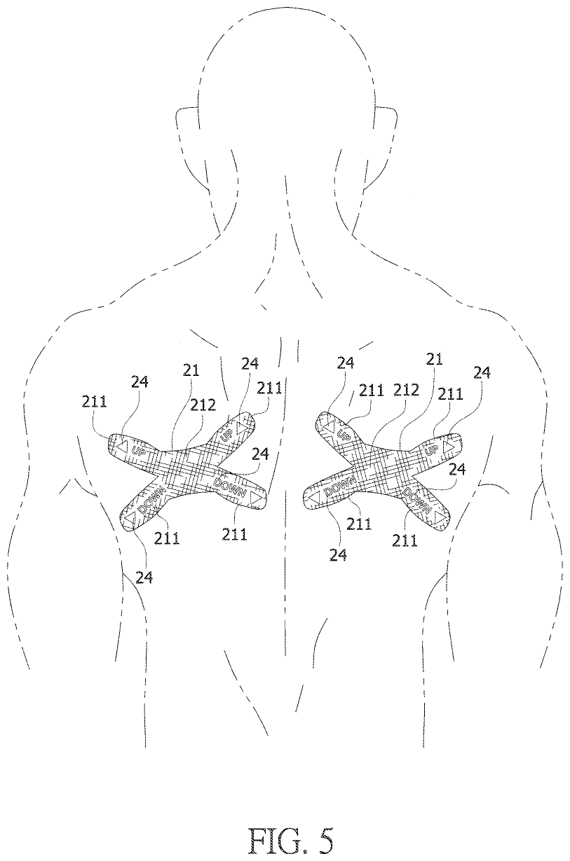

The adhesion section 211 and the positioning section 212 can be designed and implemented in different shapes to meet different needs, such as the following examples. Firstly, as shown in FIG. 3, which discloses the first embodiment, the adhesion section 211 has two portions, and is made into a “V” shape, which suits the arm; as shown in FIG. 4, which discloses the second embodiment of the invention, the adhesion section 211 has three portions, and is made into an arrow shape, like the symbol “Ψ”, to suit the shoulder; as shown in FIG. 5, which discloses the third embodiment, the adhesion section 211 has four portions, and is made into a crossing shape, like the letter “X”, to suit the back.

Moreover, the length and width of the adhesion section 211 can be designed according to actual needs. In principle, the length can be extended and the excessive length of the adhesion section 211 can be cut off using scissors. The width can also be slightly larger than the traditional sports tapes. The excessive width of the adhesion section 211 can also be narrowed using scissors; when the adhesion section 211 has two or more portions, the two portions can be implemented as equal or different lengths; moreover, the tape layer 21 can provide different thickness, elasticity, softness to meet different needs, so that users can enjoy more flexible options.

Further referring to FIG. 1 to FIG. 5, the surfaces of the adhesion section 211 and positioning section 212 of the tape layer 21 can be provided with indicative or guiding labels 24, such as an arrow shape “Δ” to indicate the direction, or English words like “UP” or “DOWN” to indicate the positions. The labels 24 can be implemented in form of stickers or printings. The sharp-color labels 24 can help consumers to operate quickly according to the indication or guidance.

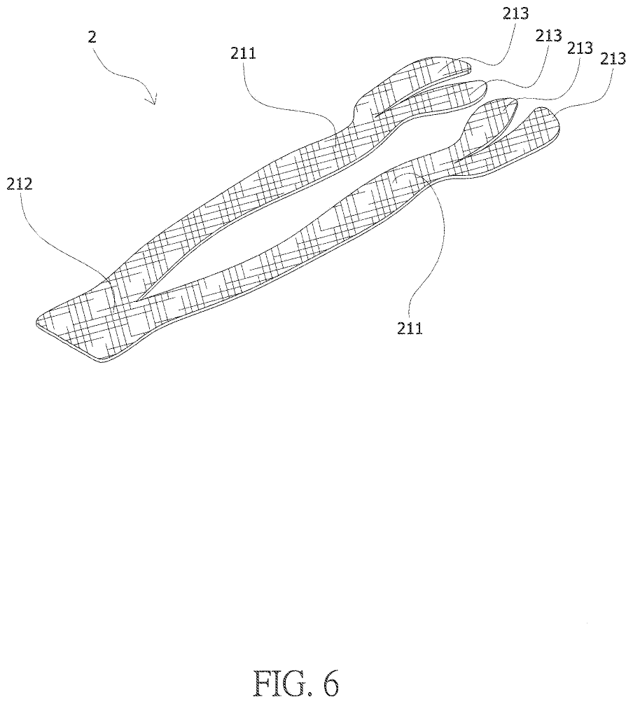

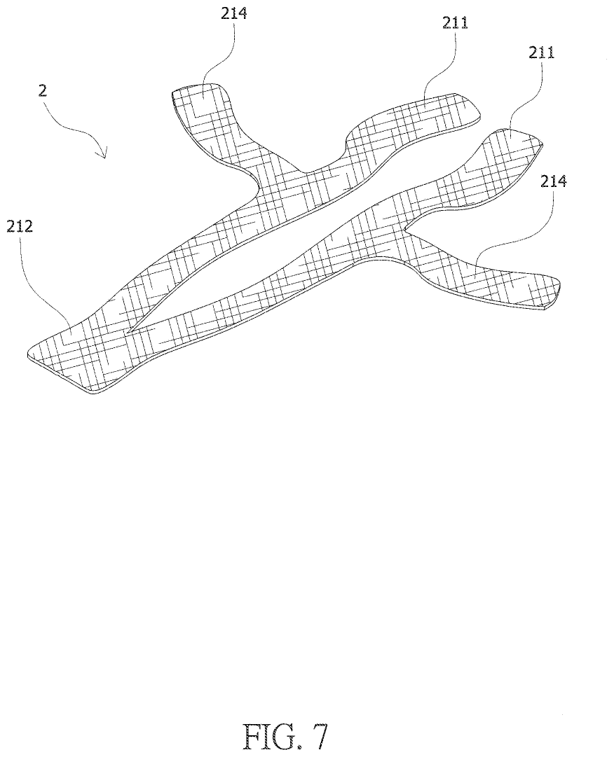

The tape body 2 can also be implemented in the following shapes. Referring to FIG. 6, the adhesion section 211 disclosed in the figure has two or more branching sections 213 on its free end; referring to FIG. 7, the adhesion section 211 disclosed in the figure has at least one extended section 214 extending from its side; the above-mentioned adhesion section 211 can also have branching sections 213 or extended sections 214 at the same time; through the above-mentioned implementations of the branching sections 213 or extended sections 214, the adhesion section 211 can be adhered to the body more stably.

In the above structure, the tape layer 21 is made of a synthetic polymer, which can be any of the following materials: polyethylene (PE), polypropylene (PP), polyvinyl chloride, PVC), ethylene vinyl acetate copolymer (EVA), polyvinyl alcohol (PVA), polyamide (Nylon), thermoplastic polyurethane (TPU), polyethylene terephthalate (PET), or any combination of two or more than two of the above materials.

The diversified implementing forms of the present invention as described above offer diversified options and applications for users. Comparing to conventional sports tapes, the present invention is more convenient to use. Moreover, the present invention is easier to use. Consumers can easily learn to use the product by themselves and no longer need to worry about the difficulty of usage.

I claim:

1. A convenient sports tape, comprising
a package body (1) and a tape body (2);
wherein the package body (1) has at least one transparent top sheet (11), a bottom sheet (12) corresponding to the top sheet (11) and connected to the top sheet (11) at an edge, and a housing space (13) formed between the top sheet (11) and the bottom sheet (12), the housing space (13) can store and clamp the tape body (2); the tape body (2) is provided with at least one adhesion section (211), and a positioning section (212) configured on one end of the adhesion section (211);
the tape body (2) has three layers, made up of a tape layer (21), an adhesive layer (22), and a releasing layer (23) of the same shape; the tape layer (21) can be configured according to the marbling of lean flesh; the adhesive layer (22) is configured on a bottom of the tape layer (21), and can be adhered to and positioned on the corresponding marbling of lean flesh of an user; the releasing layer (23) is configured on the bottom of the adhesive layer (22) in a detachable form.

2. The convenient sports tape defined in claim 1, wherein a surface of the adhesion section (211) and positioning section (212) of the tape layer (21) is configured with indicating or guiding labels (24).

3. The convenient sports tape defined in claim 1, wherein the terminal of a free end of the adhesion section (211) is formed with two or more branching sections (213).

4. The convenient sports tape defined in claim 1, wherein one side of the adhesion section (211) is formed with at least one extended section (214).

5. The convenient sports tape defined in claim 1, the tape layer (21) is made of a synthetic polymer, which can be any of the following materials: polyethylene (PE), polypropylene (PP), polyvinyl chloride (PVC), ethylene vinyl acetate copolymer (EVA), polyvinyl alcohol (PVA), polyamide (Nylon), thermoplastic polyurethane (TPU), polyethylene terephthalate (PET), or any combination of two or more than two of the above materials.
